(19) United States
(12) Patent Application Publication
(10) Pub. No.: US 2004/0068292 A1
(43) Pub. Date: Apr. 8, 2004

(54) Surgical suture material formed with needle insert mould method

(76) Inventor: Tomoaki Koseki, Chiyoda-ku (JP)

Correspondence Address: Tomoaki Koseki, 17-2, Sotokanda 2-chome, Chiyoda-ku, Tokyo, 101-0021, JP

(21) Appl. No.: 10/673,403

(22) Filed: Sep. 26, 2003

(30) Foreign Application Priority Data
Sep. 26, 2002 (JP) JP-2002-281814

(51) Int. Cl. A61B 17/06

(52) U.S. Cl. 606/224

(57) ABSTRACT

When a needle and thread are attached with caulking, the needle easily falls off, and the needle easily slips when held with a needle holder, and the processing expense is high. When a needle breaks inside the tissue, the broken part is very difficult to be removed, and creates a severe situation. Surgical knot fastening is troublesome, and the knots often get loosened. To solve these issues, a metal needle is insert-moulded to one end of resin made suture material. The resin suture material is in a sash form, the surface of the sash has a multiple number of serial projections for prevention of untwining, the other end is made in a box form, the inside of the box is formed to receive the untwine-prevention projections, and thus the suture part is gradually fastened and fixed. The manufacturing method is that the needle which is insert-moulded is a curved needle, and the sash is formed so that the needle would stand perpendicular to the sash suture material plane.

### BACKGROUND OF THE INVENTION

1. Field of the invention

This invention relates to a surgical suture material utilized during a surgery.

2. Description of the Prior Art

Majority of surgical suture material are called needle-attached thread, wherein a needle that has a vertical hole is fixed with caulking at one end of the thread. After the needle is pierced through a body tissue, the needle is cut off, and knots are fastened according to surgical fastening methods.

Needles fixed onto threads with caulking method easily fall out of place. In cases of round needles with metal body, the needles easily slip when held with a needle holder. When the needle breaks inside the body tissue, the broken part is difficult to be removed, and the situation for the patient could become very severe. Surgical knot fastening is very troublesome, and especially when suture is conducted with material with high smooth surface, the knot sometimes becomes loose and untied. Also the processing expense is high.

### SUMMARY OF THIS INVENTION

To solve the above issues, this invention proposes a metal needle that is made with insert-mould method inserted on one end of resin suture material.

The center part of the resin suture material is in a sash form, the surface of the sash has a multiple number of serial projections to prevent untwining, the other end being formed in a box form. After piercing of a body tissue for the suture is conducted, the needle is cut off, the cut section is passed through the box, the lower part of the upper portion of the inside of the box has receptors to receive the untwining projections, and thus the suture part can be fastened and fixed gradually.

The resin is used as a material which can be absorbed in the body though mechanisms such as hydrolysis and decomposition by enzymes.

The metal needle is made of insert-mould method in a curved shape, and the manufacturing method proposed is to mould the needle so that the curving direction is perpendicular to the sash form suture material plane, so that the needle would stand against the suture plane.

### DESCRIPTION OF THE PREFERRED EMBODIMENTS

Hereafter, a description of the preferred embodiments of this invention is made in reference to drawings. FIG. 1 shows one embodiment of this invention, wherein one end of sash form suture material 1 has a metal needle 2 insert-mould, one side of the inside of the sash has a multiple number of serial projections for prevention of untwining. The other end of the sash is formed in a box form 3, so after the needle is pierced through the tissue, the needle is cut off and then the cut section is passed through into box 3. The inside of the box has receptors to receive the untwine prevention projections, enabling gradual fastening and then fixing of the tissue. The needle is covered with resin up to almost its center part, so there is no concern for the needle to fall off or break, the tempering hardness is very high, and it is possible to use affordable and cost effective material such as stainless SUS420, SUS630 and such. As a curved suture needle is inserted perpendicularly to the sash plane, the resin holds the tissue in a flat way against the needle piercing plane, thus causing least invasiveness and strong fastening. The preferable material for the sash suture material and the solid body mould box part is bio-adaptable material such as polyethylene, polypropyrene and such. Also, when material such as polyglycol acid, poly lactic acid, and polydioxanone that can be absorbed within the body is utilized, the material would not be left inside the body.

As explained above, this invention enables easy and strong fastening and suture of tissues. As the needle is covered with resin, the needle does not slip so much when held with a needle holder. There is less concern of the needle falling out or breaking, the tempered hardness is high, and it is possible to use affordable and cost effective material such as stainless SUS420, SUS630 and such. As a curved suture needle is inserted perpendicularly to the sash plane, the resin holds the tissue in a flat way against the needle piercing plane, thus causing least invasiveness and strong fastening. When material that can be absorbed within the body is utilized, the material would not be left inside the body.

### BRIEF DESCRIPTION OF THE DRAWINGS

FIG. 1 is an explanatory drawing of one embodiment of a surgical suture material wherein a needle is made of insert-mould method.

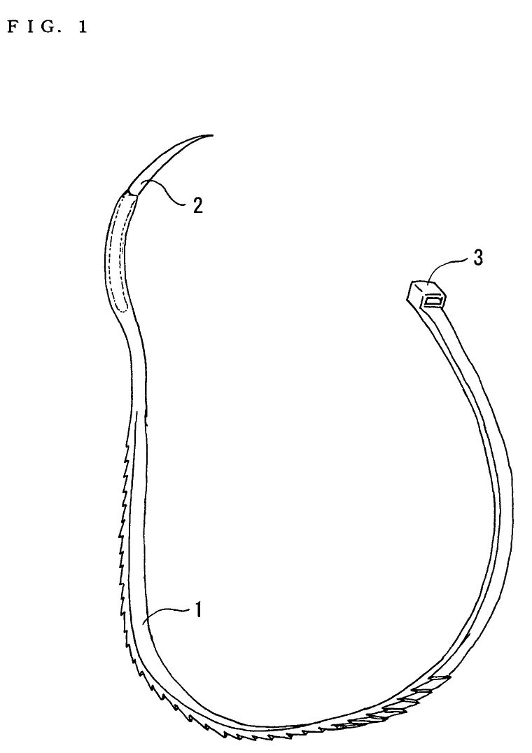

What is claimed is:

1. A suture material used during a surgery, wherein a metal needle is attached with insert-mould method inserted within resin material, attached to one end of a resin suture material.

2. A surgical suture material according to claim 1, wherein the center part of the resin suture material is in a sash form, the surface of the sash has a multiple number of serial projections formed to prevent untwining, the other end is formed in a box form, and when the needle pierces through the suture part, the needle is cut off; the cut section is passed through the box, the lower part of the upper portion of the inside of the box having receptors to receive the untwine-prevention projections, and thus the suture part can be gradually fastened and fixed.

3. A surgical suture material according to claims 1 and 2, wherein the resin is made of material that can be absorbed inside the body with mechanisms such as hydrolysis or decomposition by enzymes.

4. A manufacturing method for the above surgical suture material according to claim 3, wherein the metal needle which is made of insert-mould method made in a curved shape, and the curved direction is formed so that the needle can stand perpendicular against the suture material plane.
